(19) United States
(12) Patent Application Publication
(10) Pub. No.: US 2026/0227382 A1
(43) Pub. Date: Aug. 6, 2026

(54) SYSTEMS AND METHODS FOR DETERMINING POST-COSMETIC TREATMENT

(71) Applicant: L’Oreal, Paris (FR)

(72) Inventors: Xue LIU, Berkeley Heights, NJ (US); I-Chien LIAO, Princeton, NJ (US); Rebecca BARRESI, Robbinsville, NJ (US); Jyotsna PATURI, Cupertino, CA (US); Janet WANGARI OLIVERO, Ringoes, NJ (US)

(21) Appl. No.: 19/042,269

(22) Filed: Jan. 31, 2025

(51) Int. Cl. G01N 33/50; G16H 20/00

(52) U.S. Cl. CPC G01N 33/5008; G16H 20/00

(57) ABSTRACT

Systems, apparatuses, methods, and computer program products are disclosed for determining post-cosmetic treatment. In embodiments, methods may include receiving a biological sample from a subject corresponding to an area treated with a cosmetic treatment. Methods may include performing multi-OMICS testing on the biological sample to generate multi-OMICS data. Methods may include analyzing the multi-OMICS data, thereby to generate cell characteristics of the biological sample. Methods may include determining a difference between the cell characteristics of the biological sample and cell characteristics of a control sample. Methods may include scoring at least one cell characteristic of the biological sample based on (a) the difference and (b) on variables within the at least one cell characteristic of the biological sample. Methods may include determining the post-cosmetic treatment based on a score of the at least one cell characteristic.

### TECHNOLOGICAL FIELD

Example embodiments of the present disclosure relate generally to systems and methods for determining a post-cosmetic treatment based on analysis of multi-OMICS data of one or more biological samples corresponding to an area treated with a cosmetic treatment.

### BACKGROUND

Skin serves as the largest organ of the human body, separating a person’s internal environment from the environment and/or external world. The skin is heterogeneous from the genetic level to the tissue level, with structural components varying based on anatomical location. In the realm of dermatological research and cosmetic science, understanding the intricacies of skin responses to various treatments and procedures is crucial for developing effective skincare strategies.

Traditional approaches to evaluating cosmetic procedures and selecting complementary skincare products rely on limited data sets, typically focusing on clinical outcomes or isolated molecular markers. However, the complexity of skin biology and the diverse effects of cosmetic treatments necessitate a more comprehensive approach.

### BRIEF SUMMARY

Recent advancements in multi-OMICS technologies, including genomics, transcriptomics, proteomics, and metabolomics, have opened new avenues for understanding skin biology at unprecedented depth. These technologies, when combined with traditional clinical assessments and histological analyses, offer the potential for a more holistic understanding of skin responses to cosmetic procedures.

As noted, typical cosmetic evaluation procedures rely on limited data sets. Further, such an evaluation does not identify and/or determine a complimentary and/or effective post-cosmetic treatment.

Thus, there is felt a need to provide an integrated system that can comprehensively evaluate the effects of cosmetic devices, procedures, and/or formula on skin (e.g., a cosmetic treatment), and use this information to guide the selection of complementary, post-cosmetic treatment. Such a system would enable more personalized and effective cosmetic solutions, optimizing treatment outcomes and addressing specific skin concerns with greater precision. Such systems and methods may enable prompt identification or determination of certain or selected cellular characteristics of skin samples with a cosmetic treatment applied thereto. Such an identification or determination may include analyzing OMICS or multi-OMICS data associated with biological samples (for example ex vivo skin and/or clinical biopsies, among other types of samples) to produce cell characteristics via an algorithm or circuitry (for example, an analysis algorithm and/or a machine learning model) Another algorithm (for example, a ranking or scoring algorithm and/or another machine learning model) may be utilized to determine a ranking or score of the cell characteristics. Finally, utilizing that ranking, the systems and methods described herein may include determining a post-cosmetic treatment. Such a treatment may include utilizing a selected professional tool, device, or equipment; utilizing a selected home-use tool, device, or equipment; utilizing a selected cosmetic product or formula; and/or ceasing or continuing use of the cosmetic treatment. Such determinations, as noted, may be based on the ranking or scoring of the cell characteristics. The scoring and ranking may additionally indicate the efficacy of the cosmetic treatment. Further, such determinations may be additionally based on historical data, subject data, product or formula data, and/or tool, device, or equipment data, among other data.

Thus, subjects and/or users can quickly and easily determine a compatible and complimentary post-cosmetic treatment. Further, in embodiments, the determined post-cosmetic treatment may be selected to boost or improve the efficacy of the previously utilized cosmetic treatment and/or may be selected specifically for a particular subject or user.

Accordingly, an embodiment of the disclosure is directed to a method for determining post-cosmetic treatment. The method may include receiving a biological sample from a subject corresponding to an area treated with a cosmetic treatment. The method may include performing multi-OMICS testing on the biological sample to generate multi-OMICS data. The method may include analyzing the multi-OMICS data, thereby to generate cell characteristics of the biological sample. The method may include determining a difference between the cell characteristics of the biological sample and cell characteristics of a control sample. The method may include scoring at least one cell characteristic of the biological sample based on (a) the difference and (b) on variables within the at least one cell characteristic of the biological sample. The method may include determining the post-cosmetic treatment based on a score of the at least one cell characteristic.

In an embodiment, the cosmetic treatment may include use of one or more of a professional device, a home-use device, or a topical treatment. In another embodiment, the multi-OMICS testing includes one or more of histology and immunohistochemistry, spatial transcriptomics, bulk RNA sequencing, or protein expression analysis. In a further embodiment, the method may include analyzing the multi-OMICS data comprises one or more of overlaying spatial gene expressions with histology images. The method may include performing differential expression analysis between the biological sample and the control. The method may include analyzing gene ontology and pathway enrichment; or integrating transcriptomics, proteomics, and clinical data.

In another embodiment, the analysis of the multi-OMICS data may include applying the multi-OMICS data to a trained machine learning model thereby to predict treatment outcomes.

Another embodiment of the disclosure is directed to an apparatus for determining post-cosmetic treatment. The apparatus may include a multi-OMICS testing circuitry. The multi-OMICS testing circuitry may be configured to receive a biological sample from a subject corresponding to an area applied with a cosmetic treatment over a selected time. The multi-OMICS testing circuitry may be configured to execute multi-OMICS testing on the biological sample to generate multi-OMICS data. The apparatus may include a scoring circuitry. The scoring circuitry may be configured to apply the multi-OMICS data to one or more of (a) one or more analysis algorithms or (b) a trained machine learning model, thereby to generate cell characteristics of the biological sample. The scoring circuitry may be configured to determine a difference between the cell characteristics of the biological sample and cell characteristics of a control. The scoring circuitry may be configured to score at least one cell characteristic of the biological sample based on (a) the cell characteristics of the biological sample and (b) the difference. The scoring circuitry may be configured to determine the post-cosmetic treatment based on a score of the at least one cell characteristic.

In an embodiment, the score may indicate an efficacy of the cosmetic treatment in comparison to the control.

In another embodiment, the scoring circuitry may be configured to, prior to generation of a score for each of the cell characteristics of the biological sample, classify cell characteristics into net upregulated or net downregulated signaling pathways; and wherein the score for each of the cell characteristics of the biological sample is further based on classification of the cell characteristics.

In an embodiment, the control may include one or more of an untreated sample, a standard, or a comparable standard. In another embodiment, the post-cosmetic treatment may include a personalized cosmetic treatment regimen.

In an embodiment, each of the cell characteristics correlate to selected cellular attributes, and wherein the cellular attributes comprise one or more of lipid metabolism, extracellular matrix organization, cellular stress response, cellular detoxification, immune responses, or inflammatory response.

Another embodiment of the disclosure is directed to a computer program product for determining post-cosmetic treatment, the computer program product comprising a non-transitory machine-readable storage medium storing software instructions. The software instructions, when executed, may cause an apparatus to execute multi-OMICS testing on a biological sample from a subject corresponding to a cosmetic treatment occurring over a selected time, thereby to generate multi-OMICS data. The software instructions, when executed, may cause the apparatus to apply the multi-OMICS data to one or more analysis instructions, thereby to generate cell characteristics of the biological sample. The software instructions, when executed, may cause the apparatus to determine a difference between the cell characteristics of the biological sample and cell characteristics of an untreated sample or standard. The software instructions, when executed, may cause the apparatus to score the cell characteristics of the biological sample based on (a) the cell characteristics of the biological sample and (b) the difference. The software instructions, when executed, may cause the apparatus to determine the post-cosmetic treatment based on a score of at least one of the cell characteristics.

In another embodiment, the one or more analysis instructions may include one or more of (a) multi-OMICS analysis algorithms or (b) a trained machine learning algorithm. In another embodiment, the post-cosmetic treatment may include one or more of a use of a selected device or a use of a selected cosmetic.

The foregoing brief summary is provided merely for purposes of summarizing example embodiments illustrating some aspects of the present disclosure. Accordingly, it will be appreciated that the above-described embodiments are merely examples and should not be construed to narrow the scope of the present disclosure in any way. It will be appreciated that the scope of the present disclosure encompasses many potential embodiments in addition to those summarized above, some of which will be described in further detail below.

### BRIEF DESCRIPTION OF THE FIGURES

Having described certain example embodiments of the present disclosure in general terms above, reference will now be made to the accompanying drawings, which are not necessarily drawn to scale. Some embodiments may include fewer or more components than those shown in the figures.

FIG. 1A and FIG. 1B illustrate systems in which some example embodiments may be used for determining a post-cosmetic treatment.

FIG. 2 illustrates a schematic block diagram of example circuitry embodying a device that may perform various operations in accordance with some example embodiments described herein.

FIG. 3A and 3B illustrate example flowcharts for determining post-cosmetic treatment, in accordance with some example embodiments described herein.

### DETAILED DESCRIPTION

Some embodiments of the present disclosure will now be described more fully hereinafter with reference to the accompanying figures, in which some, but not all, embodiments of the disclosures are shown. Indeed, these disclosures may be embodied in many different forms and should not be construed as limited to the embodiments set forth herein; rather, these embodiments are provided so that this disclosure will satisfy applicable legal requirements.

The term “computing device” is used herein to refer to any one or all of programmable logic controllers (PLCs), programmable automation controllers (PACs), industrial computers, desktop computers, personal data assistants (PDAs), laptop computers, tablet computers, smart books, palm-top computers, personal computers, smartphones, wearable devices (such as headsets, smartwatches, or the like), and similar electronic devices equipped with at least a processor and any other physical components necessarily to perform the various operations described herein. Devices such as smartphones, laptop computers, tablet computers, and wearable devices are generally collectively referred to as mobile devices.

The term “server” or “server device” is used to refer to any computing device capable of functioning as a server, such as a master exchange server, web server, mail server, document server, or any other type of server. A server may be a dedicated computing device or a server module (e.g., an application) hosted by a computing device that causes the computing device to operate as a server. A server module (e.g., server application) may be a full function server module, or a light or secondary server module (e.g., light or secondary server application) that is configured to provide synchronization services among the dynamic databases on computing devices. A light server or secondary server may be a slimmed-down version of server type functionality that can be implemented on a computing device, such as a smart phone, thereby enabling it to function as an Internet server (e.g., an enterprise e-mail server) only to the extent necessary to provide the functionality described herein.

As used herein, a “non-transitory machine-readable storage medium” or “memory” may be any electronic, magnetic, optical, or other physical storage apparatus to contain or store information such as executable instructions, data, and the like. For example, any machine-readable storage medium described herein may be any of random access memory (RAM), volatile memory, non-volatile memory, flash memory, a storage drive (e.g., hard drive), a solid state drive, any type of storage disc, and the like, or a combination thereof. The memory may store or include instructions executable by the processor.

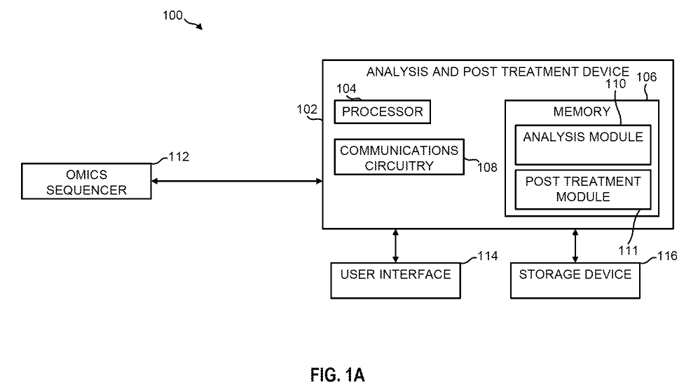

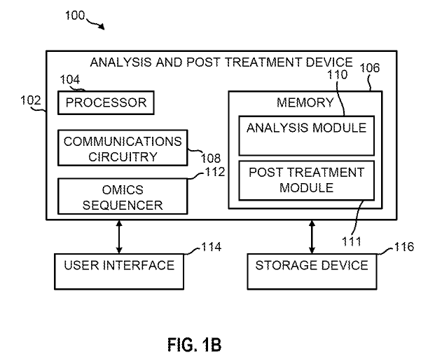

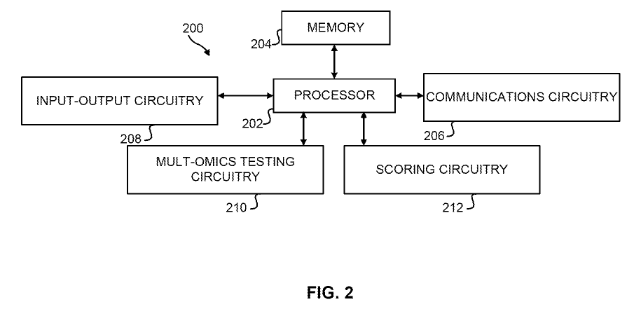

As used herein, a “processor” or “processing circuitry” may include, for example one processor or multiple processors included in a single device or distributed across multiple computing devices. The processor (such as, processor 104 and processor 202 shown in FIGS. 1A through 2) may be at least one of a central processing unit (CPU), a semiconductor-based microprocessor, a graphics processing unit (GPU), a field-programmable gate array (FPGA) to retrieve and execute instructions, a real time processor (RTP), other electronic circuitry suitable for the retrieval and execution instructions stored on a machine-readable storage medium, or a combination thereof.

As noted above, methods, apparatuses, systems, and computer program products are described herein that provide scores or rankings of cell characteristics of one or more biological samples that a cosmetic treatment has been applied to and, utilizing those scores or rankings and other data related to the one or more biological samples, a determination of a complimentary, compatible, and effective post-cosmetic treatment may be performed. Traditional approaches to evaluating cosmetic procedures and selecting complementary skincare products and/or other treatments, including the continued use of the previous cosmetic procedure, rely on limited data sets, such methods focusing on clinical outcomes or isolated molecular markers. The complexity of skin biology and the diverse effects of cosmetic treatments (such as, for example, topical, formula based treatments and/or device based treatments) necessitate a more comprehensive approach. In addition, typically, such analysis and/or interpretation includes some level of bias, potentially skewing the end interpretation. Thus, the best post-cosmetic treatment or any post-cosmetic treatment may not be selected by the end of such processes. In other words, typical analysis merely provides some output indicating a narrow interpretation of treatment efficacy and does not include some output indicating potential complimentary post-cosmetic treatments and/or whether continued treatment, at varying levels (e.g., device intensity and/or formula concentrations) could provide some benefit.

In contrast to these conventional techniques for analyzing use of cosmetic treatments, the present disclosure describes utilizing multi-OMICS analysis of biological samples (for example, ex vivo skin samples and/or clinical biopsies) that a cosmetic treatment has been applied thereto and determining, based on that analysis, a post-cosmetic treatment. The cosmetic treatment and post-cosmetic treatment may include application of an ointment or other type of cosmetic and/or use of a cosmetic device or apparatus. Post-cosmetic treatment may also include varied use of the cosmetic treatment. Further, the ointment or other type of cosmetic treatment may include one or more of a plurality of active agents. The plurality of active agents may include, for example, naturally occurring peptides or other types of agents. The devices may include one or more of a device that uses radiofrequency, fractional lasers, ablative lasers, non-ablative lasers, or micro needling. Further, the cosmetic device may include a professional device (for example, a device utilized at a dermatologist’s office) or a home-use device (for example, a hand-held device designed for use by a non-professional). In an embodiment, the biological samples may be received by an apparatus including an analyzer and/or the software instructions described herein. In another embodiment, the apparatus may receive the multi-OMICS analysis data from a corresponding device. In yet another embodiment, the apparatus may be positioned at a professional’s location and utilized by the professional, while in other embodiments, the apparatus may be configured for home-use.

As noted above, the systems and methods described herein may utilize multi-OMICS testing and analysis. The multi-OMICS testing and analysis may include genomics, transcriptomics, proteomics, and metabolomics, among other types of tests and/or analysis. Once multi-OMICS testing has been performed, that multi-OMICS data may be analyzed to produce and/or generate cell characteristics. Further, the cell characteristics may be ranked or scored. Finally, that ranking or scoring may be utilized, along with, in some embodiments, other variables of the cell characteristics and/or biological sample, to determine a complementary, post-cosmetic treatment. In an embodiment, an algorithm, analysis algorithm, mathematical model, and/or machine learning model may be utilized and/or configured to perform any of the steps described herein Further, such software instructions may utilize historical data related to prior testing of a variety of subjects utilizing one or more different cosmetic treatments, post-cosmetic treatments, and/or the resulting outcome of the cosmetic treatment and/or post-cosmetic treatment.

Accordingly, the present disclosure sets forth systems, methods, and apparatuses that provide custom tailored and quickly generated post-cosmetic treatments based on multi-OMICS analysis of biological samples. There are many advantages of these and other embodiments described herein. For instance, the resulting post-cosmetic treatment may be specific for a user or subject. Further, such a result may be based on extensive historical data, thus providing a more complimentary and efficient post-cosmetic treatment. In addition, the systems and methods described herein may provide insight into the effectiveness of certain cosmetic treatments as standalone treatments and in conjunction with a prescribed post-cosmetic treatment.

Although a high level explanation of the operations of example embodiments has been provided above, specific details regarding the configuration of such example embodiments are provided below.

Example embodiments described herein may be implemented using any of a variety of computing devices or servers. To this end, FIG. 1A and FIG. 1B illustrate an example environment within which embodiments of the present disclosure may operate. As illustrated, a post-cosmetic treatment determination system 100 may include an analysis and post treatment device 102. The analysis and post treatment device 102 may include a processor 104, a memory 106, and/or a communications circuitry 108. The memory 106 may include instructions and/or other software or algorithms, such as an analysis module 110 and/or a post treatment module 111 or a post-cosmetic treatment module. The analysis and post treatment device 102 may connect, via the communication circuitry 108 and/or a communications network, to an OMICS sequencer 112, a user interface 114, and/or a storage device 116, among other devices and/or components. As illustrated and/or in other implementations, the post-cosmetic treatment determination system 100, and any constituent device(s) and/or storage device(s) 116 may receive and/or transmit information via a communications network (for example, the internet and/or an intranet) with any number of other devices.

The analysis and post treatment device 102 may be implemented as one or more servers, which may or may not be physically proximate to other components of the post-cosmetic treatment determination system 100. Furthermore, some components of the analysis and post treatment device 102 may be physically proximate to the other components of the post-cosmetic treatment determination system 100 while other components are not. The analysis and post treatment device 102 may receive, process, generate, and transmit data, signals, and electronic information to facilitate the operations of the post-cosmetic treatment determination system 100. Particular components of the analysis and post treatment device 102 are described in greater detail below with reference to apparatus 200 in connection with FIG. 2. In another example, the analysis and post treatment device 102 may be implemented as a computing device or personal computer (such as a laptop or desktop computer). In yet another example, the analysis and post treatment device 102 may be implemented as a singular medical device. In such an example, the OMICS sequencer 112 may be integrated within or be a part of the analysis and post treatment device 102 (as shown in FIG. 1B). Further, in such examples, the analysis and post treatment device 102 may be a home-use device and/or positioned at a doctor’s, physician’s, and/or other medical professional’s office.

As noted, the memory 106 may include instructions and/or other algorithms. Such instructions, when executed by the processor 104, may control various functions and/or aspects of the analysis and post treatment device 102. For example, a user may, via the user interface 114, initiate analysis of a biological sample. In another example, initiation of the analysis may occur based on entry of a biological sample into the OMICS sequencer 112 or upon reception of OMICS data from an external device. Such an initiation may occur via the analysis module 110. In other words, upon occurrence of some condition (for example, user initiation or reception of a biological sample), the analysis module 110 may initiate analysis of the biological sample. Upon such an initiation, the analysis and post treatment device 102 may cause the OMICS sequencer 112 to analyze one or more biological samples. Such biological samples may include an ex vivo skin sample, a clinical biopsy sample, and/or cells obtained in some other way (for example, cells or biological samples may be obtained from a subject via a mail-in-kit or via other methods). Each biological sample may have been previously treated with a selected cosmetic treatment. Cosmetic treatments may include, for example, application of a cosmetic (such as a topical ointment), use of a cosmetic device (such as a professional or home use device that utilizes radiofrequency, fractional lasers, ablative lasers, non-ablative lasers, or micro needling), or some combination thereof. Such information (e.g., the treatment of a biological sample with a selected cosmetic treatment) may be recorded or entered into the analysis and post treatment device 102, along with other factors or variables, such as subject information (for example, clinical variables (including, but not limited to, age, weight, height, gender, skin type, hair type, other sample or cell type, and/or medical history) length of time the cosmetic treatment was utilized, amount and/or intensity of the cosmetic treatment used, and/or other factors related to the cosmetic treatment). The OMICS sequencer 112 may produce data corresponding to one or more characteristics, gene expressions (for example, enrichment, depletion, and/or other types of gene expressions), and/or other factors of the plurality of cells. Such data may be referred to as multi-OMICS data. The OMICS sequencer 112 may produce a cell-by-gene matrix, gene expressions, and/or other data in various formats. For example, the OMICS sequencer 112 may generate skin barrier cell values upon analysis (e.g., via the analysis module 110). In another embodiment, the analysis and post treatment device 102 may receive or obtain data (such as multi-OMICS data) from the storage device 116, via the user interface 114, and/or from an external device in communication with the analysis and post treatment device 102 via the communications circuitry 108. In another embodiment, the OMICS sequencer 112 may be included in or with or integrated into or with the analysis and post treatment device 102, as illustrated in FIG. 1B. In yet another embodiment, the analysis and post treatment device 102 may not include or connect to an OMICS sequencer 112 and may receive OMICS data from an external device via the communications circuitry 108. In an embodiment, the one or more characteristics may include one or more of differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health. In another embodiments, the cell characteristics may correlate to selected cellular attributes and the cellular attributes may include one or more of lipid metabolism, extracellular matrix organization, cellular stress response, cellular detoxification, immune responses, or inflammatory response. In another embodiment, the multi-OMICS data may include overlaying spatial gene expressions with histology images; performing differential expression analysis between the biological sample and the control; analyzing gene ontology and pathway enrichment; or integrating transcriptomics, proteomics, and/or clinical data, among other OMICS data.

As noted, upon reception or generation of multi-OMICS data, the analysis and post treatment device 102 may analyze, for example, via an analysis algorithm and/or machine learning model, the multi-OMICS data via the analysis module 110 to produce or generate one or more characteristics and/or one or more cell characteristics. Such an analysis algorithm or machine learning model may utilize historical data to interpret and/or transform the multi-OMICS data into the sets of one or more characteristics and/or one or more cell characteristics. Further, the resulting one or more characteristics and/or one or more cell characteristics may be based on the type of sample analyzed. For example, certain characteristics (for example, skin barrier values, inflammation values, and pigmentation characteristics, among others) may be determined for skin samples, while others may be utilized for hair samples.

Once the one or more characteristics and/or one or more cell characteristics are available, the post treatment module 111 may then determine the difference between those characteristics and characteristics of a control sample, of another biological sample, of an untreated sample, of a standard, or of a comparable standard. The post treatment module 111 may then score or rank each of the characteristics, based on those differences as well as other variables, and, based on those scores and/or other variables of the cell characteristics, determine a post treatment or post-cosmetic treatment. For example, if a certain aspect of one characteristic varies greatly and/or has a selected score that exceeds or is less than a selected range, then the post treatment module 111 may determine that a selected post-cosmetic treatment should be prescribed in conjunction with or rather than the previous cosmetic treatment. In another embodiment, the post-cosmetic treatment may include a personalized cosmetic treatment regimen. In another embodiment, the post treatment module 111 may determine that a cosmetic treatment should be continued, based on the characteristics. In a further embodiment, the post treatment module 111 may determine that the post-cosmetic treatment includes continued use of the cosmetic treatment at a varying level (e.g., using an increased or decreased amount of a cosmetic or varying the intensity, the length of time of use, and/or some other variable of a cosmetic treatment). In embodiments, the post treatment module 111 my utilize a machine learning algorithm to determine a post-cosmetic treatment. For example, the machine learning algorithm may be trained using data including various treatments and the subsequent affects. Further, data including various combinations of treatments may be included in such training sets. An input to such a trained machine learning model may include the characteristics, the score of the characteristics, the type of treatment, and/or subject data. Once the data is input, the trained machine learning model may then output a post-cosmetic treatment that is complementary to the cosmetic treatment and/or effective based on the previous cosmetic treatment. Further, such a model can be refined based on subsequent use and results of the post-cosmetic treatment.

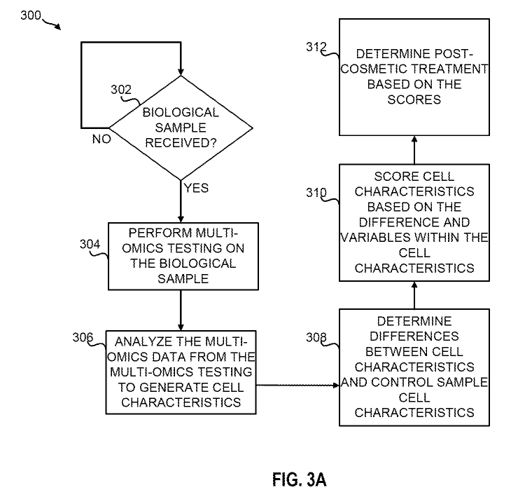

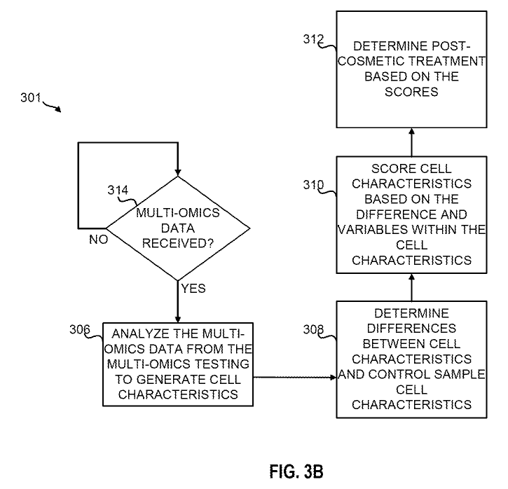

The analysis and post treatment device 102 of the post-cosmetic treatment determination system 100 (described previously with reference to FIGS. 1A and 1B) may be embodied by one or more computing devices or servers, shown as apparatus 200 in FIG. 2. As illustrated in FIG. 2, the apparatus 200 may include a processor 202, a memory 204, a communications circuitry 206, input-output circuitry 208, multi-OMICS testing circuitry 210, and/or scoring circuitry 212, each of which will be described in greater detail below. While the various components are only illustrated in FIG. 2 as being connected with processor 202, it will be understood that the apparatus 200 may further comprises a bus (not expressly shown in FIG. 2) for passing information amongst any combination of the various components of the apparatus 200. The apparatus 200 may be configured to execute various operations described above in connection with FIGS. 1A-1B and below in connection with FIGS. 3A and 3B.

The processor 202 (and/or co-processor or any other processor assisting or otherwise associated with the processor) may be in communication with the memory 204 via a bus for passing information amongst components of the apparatus. The processor 202 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 200, remote or “cloud” processors, or any combination thereof.

The processor 202 may be configured to execute software instructions stored in the memory 204 or otherwise accessible to the processor (e.g., software instructions stored on a memory 106, as illustrated in FIGS. 1A-1B). In some cases, the processor may be configured to execute hard-coded functionality. As such, whether configured by hardware or software methods, or by a combination of hardware with software, the processor 202 represent an entity (such as, physically embodied in circuitry) capable of performing operations according to various embodiments of the present invention while configured accordingly. Alternatively, as another example, when the processor 202 is embodied as an executor of software instructions, the software instructions may specifically configure the processor 202 to perform the algorithms and/or operations described herein when the software instructions are executed.

Memory 204 is non-transitory and may include, for example, one or more volatile and/or non-volatile memories. In other words, for example, the memory 204 may be an electronic storage device (e.g., a computer readable storage medium). The memory 204 may be configured to store information, data, content, applications, software instructions, or the like, for enabling the apparatus to carry out various functions in accordance with example embodiments contemplated herein.

The communications circuitry 206 may be any means such as a device or circuitry embodied in either hardware or a combination of hardware and software that is configured to receive and/or transmit data from/to a network and/or any other device, circuitry, or module in communication with the apparatus 200. In this regard, the communications circuitry 206 may include, for example, a network interface for enabling communications with a wired or wireless communication network. For example, the communications circuitry 206 may include one or more network interface cards, antennas, buses, switches, routers, modems, and supporting hardware and/or software, or any other device suitable for enabling communications via a network. Furthermore, the communications circuitry 206 may include the processing circuitry for causing transmission of such signals to a network or for handling receipt of signals received from a network.

The apparatus 200 may include input-output circuitry 208 configured to provide output to a user and, in some embodiments, to receive an indication of user input. It will be noted that some embodiments will not include input-output circuitry 208, in which case user input may be received via a separate device such as a client device or user interface included in apparatus 200. The input-output circuitry 208 may comprise a user interface, such as a display, and may further comprise the components that govern use of the user interface, such as a web browser, mobile application, dedicated client device, or the like. In some embodiments, the input-output circuitry 208 may include a keyboard, a mouse, a touch screen, touch areas, soft keys, a microphone, a speaker, and/or other input/output mechanisms. The input-output circuitry 208 may utilize the processor 202 to control one or more functions of one or more of these user interface elements through software instructions (e.g., application software and/or system software, such as firmware) stored on a memory (e.g., memory 204) accessible to the processor 202.

In addition, the apparatus 200 may further comprise or include a multi-OMICS testing circuitry 210 that, in an embodiment, may initiate analysis of cellular or biological samples each applied with a cosmetic treatment for a selected period of time to produce multi-OMICS data. In another embodiment, the multi-OMICS testing circuitry 210 may include equipment or devices that perform the cellular analysis, such as transcriptomics and/or other tests to produce various gene expressions for each biological sample. In yet another embodiment, the multi-OMICS testing circuitry 210 may generate or determine a cell-by gene matrix or other types of formatted characteristic data based on data received from an analyzer or sequencer, such as an OMICS sequencer, and/or from another device or storage device. The multi-OMICS testing circuitry 210 may utilize processor 202, memory 204, or any other hardware component included in the apparatus 200 to perform these operations, as described in connection with FIGS. 1A-1B above and FIGS. 3A-3B below. The multi-OMICS testing circuitry 210 may further utilize communications circuitry 206 to gather data from a variety of sources (such as, a storage device 116 or, as noted, an OMICS sequencer 112) and may utilize input-output circuitry 208 to receive data from a user (such as data related to cell analysis).

In addition, the apparatus 200 further comprises a scoring circuitry 212 that may score each of the cell characteristics determined by the multi-OMICS testing circuitry 210 . The scoring circuitry 212 may utilize one or more statistical models and/or machine learning models to perform such a scoring and/or ranking. The scoring circuitry 212 may process or utilize a cell-by-gene matrix to generate the scores and/or ranking. In such an embodiment, the scoring circuitry 212 may first convert the cell-by-gene matrix into an array. The scoring circuitry 212 may then remove the first column in the array, the first column including, in an example, the agent identities. Next, the scoring circuitry 212 may generate an average of the values in the array and then define the number of instances in the array. The scoring circuitry 212 may then use a statistical model, such as the Wilcoxon sign-rank test, to generate the score and/or rank. Based on the scores and/or ranking, the scoring circuitry 212 may determine a post-cosmetic treatment or post treatment. The scoring circuitry 212 may include or comprise a trained machine learning model that is trained to determine a post treatment or post-cosmetic treatment based on the scored and/or ranked characteristics, as well as, in other embodiments, other variables associated with the biological sample. The scoring circuitry 212 may utilize processor 202, memory 204, or any other hardware component included in the apparatus 200 to perform these operations, as described in connection with FIGS. 1A-1B above and FIGS. 3-4 below. The scoring circuitry 212 may further utilize communications circuitry 206 to gather data from a variety of sources (such as, from the multi-OMICS testing circuitry 210, storage device 116, and/or, as noted, an OMICS sequencer 112) and may utilize input-output circuitry 208 to receive data from a user (for example, the scoring circuitry 212 may receive data and/or a cell-by gene matrix from a user interface or other device).

Although components 202-212 are described in part using functional language, it will be understood that the particular implementations necessarily include the use of particular hardware. It should also be understood that certain of these components 202-212 may include similar or common hardware. For example, the multi-OMICS testing circuitry 210 and scoring circuitry 212 may each at times leverage use of the processor 202, memory 204, communications circuitry 206, or input-output circuitry 208, such that duplicate hardware is not required to facilitate operation of these physical elements of the apparatus 200 (although dedicated hardware elements may be used for any of these components in some embodiments, such as those in which enhanced parallelism may be desired). Use of the terms “circuitry,” and “engine” with respect to elements of the apparatus therefore shall be interpreted as necessarily including the particular hardware configured to perform the functions associated with the particular element being described. Of course, while the terms “circuitry” and “engine” should be understood broadly to include hardware, in some embodiments, the terms “circuitry” and “engine” may in addition refer to software instructions that configure the hardware components of the apparatus 200 to perform the various functions described herein.

Although the multi-OMICS testing circuitry 210 and the scoring circuitry 212 may leverage processor 202, memory 204, communications circuitry 206, or input-output circuitry 208 as described above, it will be understood that any of these elements of apparatus 200 may include one or more dedicated processor, specially configured field programmable gate array (FPGA), or application specific interface circuit (ASIC) to perform its corresponding functions, and may accordingly leverage processor 202 executing software stored in a memory (such as, memory 204), or memory 204, communications circuitry 206 or input-output circuitry 208 for enabling any functions not performed by special-purpose hardware elements. In all embodiments, however, it will be understood that the multi-OMICS testing circuitry 210 and the scoring circuitry 212 are implemented via particular machinery designed for performing the functions described herein in connection with such elements of apparatus 200.

In some embodiments, various components of the apparatus 200 may be hosted remotely (such as, by one or more cloud servers) and thus need not physically reside on the corresponding apparatus 200. Thus, some or all of the functionality described herein may be provided by third party circuitry. For example, a given apparatus 200 may access one or more third party circuitries via any sort of networked connection that facilitates transmission of data and electronic information between the apparatus 200 and the third party circuitries. In turn, that apparatus 200 may be in remote communication with one or more of the other components describe above as comprising the apparatus 200.

As will be appreciated based on this disclosure, example embodiments contemplated herein may be implemented by an apparatus 200. Furthermore, some example embodiments may take the form of a computer program product comprising software instructions stored on at least one non-transitory computer-readable storage medium (for example, memory 204). Any suitable non-transitory computer-readable storage medium may be utilized in such embodiments, some examples of which are non-transitory hard disks, CD-ROMs, flash memory, optical storage devices, and magnetic storage devices. It should be appreciated, with respect to certain devices embodied by apparatus 200 as described in FIG. 2, that loading the software instructions onto a computing device or apparatus produces a special-purpose machine comprising the means for implementing various functions described herein.

Having described specific components of example apparatus 200, example embodiments of the present disclosure are described below in connection with a series of flowcharts.

Turning to FIGS. 3A and 3B, example flowcharts are illustrated that contain example operations (e.g., method 300 and method 301) implemented by example embodiments described herein. The operations illustrated in FIGS. 3A and 3B may, for example, be performed by the post-cosmetic treatment determination system 100 and/or the analysis and post treatment device 102 of the post-cosmetic treatment determination system 100 shown in FIGS. 1A-1B, which may in turn be embodied by an apparatus 200, which is shown and described in connection with FIG. 2. To perform the operations described below, the apparatus 200 may utilize one or more of processor 202, memory 204, communications circuitry 206, input-output circuitry 208, multi-OMICS testing circuitry 210, scoring circuitry 212, and/or any combination thereof. It will be understood that user interaction with the post-cosmetic treatment determination system 100 may occur directly via input-output circuitry 208, or may instead be facilitated by a user interface 114, as shown in FIGS. 1A-1B, and which may have similar or equivalent physical componentry facilitating such user interaction. The order in which the operations are described is not intended to be construed as a limitation, and any number of the described blocks may be combined in any order and/or in parallel to implement the methods.

At block 302, an OMICS sequencer 112 may wait for reception of a biological sample. As described herein the OMICS sequencer 112 may be included in or may be separate from an analysis and post treatment device 102. The analysis and post treatment device 102 may initiate analysis of the biological sample or the OMICS sequencer 112 may automatically begin analysis based upon reception of the biological sample.

In an embodiment, the OMICS sequencer 112 may receive the biological sample via or from a clinical trial and/or from a medical professional. In another embodiment, the OMICS sequencer 112 may receive the biological sample via a mail-in-kit and/or via collection from the medical professional. For example, a user may purchase a skin analysis kit, gather a skin sample of a treated area, and package and send the sample to a designated location (e.g., where the OMICS sequencer 112 may be positioned). In another example, a user may visit a medical professional, such as a dermatologist, and provide a sample directly to the professional. In yet another example, a user may visit a cosmetic retailer and provide a sample there.

In yet another example, reception of a biological sample may include the following steps. For example, after application of an in vivo energetic or cosmetic treatment or procedure, a user, doctor, or other medical professional may perform a clinical grading assessment of the subject’s skin post-procedure. After treatment, a sample of the treated portion of the subject’s skin may be removed for testing (e.g., testing the efficacy of the treatment and/or determining a complementary treatment. Further, after treatment, a sample of an untreated portion of the subject’s skin may be removed for testing, to compare the results of tests against the treated portion of skin.

Once the analysis and post treatment device 102 receives a biological sample (e.g., a treated portion of the subject’s skin and, in an additional embodiment, the untreated portion of the subject’s skin), at block 304, the OMICS sequencer 112 may perform multi-OMICS testing on the biological sample to produce or generate multi-OMICS data. Multi-Omics data may include overlaying spatial gene expressions with histology images; performing differential expression analysis between the biological sample and the control; analyzing gene ontology and pathway enrichment; or integrating transcriptomics, proteomics, and clinical data; among other types of data. The multi-OMICS testing may include histology and immunohistochemistry, spatial transcriptomics, bulk RNA sequencing, or protein expression analysis, among other OMICS testing. Alternatively, at block 314 in method 301, the analysis and post treatment device 102 may wait until multi-OMICS data is received.

At block 306, the analysis and post treatment device 102 may analyze the multi-OMICS data to produce cell characteristics. Such an analysis may be performed via an analysis algorithm and/or machine learning model stored within the analysis and post treatment device 102. Such a machine learning model may be trained with a data set including a plurality of multi-OMICS data and corresponding cell characteristics in a selected format. In embodiments, analysis of the samples may include characterizing certain aspects of analyzed samples corresponding multi-OMICS data. For example, the sample’s layer morphology (e.g., vacuolization, DEJ, and/or collagen organization, among other characteristics), protein (via immunoassay, spectrophotometry, and/or chromatography, among other characteristics), and/or expression of multiple genes (potentially including spatial variation) may be characterized.

At block 308, the analysis and post treatment device 102 may determine the differences between the cell characteristic and a control set of cell characteristics, desired or selected cell characteristics, cell characteristics of an untreated sample (for example, an untreated sample of the subject’s skin or an untreated sample of another subject’s skin), a standard, or a comparable standard. The desired or selected cell characteristics may include one or more factors or characteristics that a meet a target characteristic (e.g., differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health, among other characteristics), as indicated by a medical professional and/or a subject. In yet another embodiment, the analysis and post treatment device 102 may compare the characterization of the samples (e.g., the treated sample and untreated sample) expression of multiple genes to determine or assess the upregulation and/or downregulation related to the energetic and/or cosmetic treatment and/or procedure. In another embodiment, determining the differences may include classifying and grouping selected characteristics, for example, classifying and grouping gene expressions into net regulated and/or downregulated signaling pathways.

At block 310, based on the cell characteristics and the differences determined in block 308, the analysis and post treatment device 102 may determine scores for each of the cell characteristics. Such scores may be determined via a statistical model and/or machine learning model trained to produce scores for cell characteristics. In an embodiment, prior to generating the score, the analysis and post treatment device 102 may classify cell characteristics into net upregulated or net downregulated signaling pathways. In such embodiments, the score may be further based on these classifications.

At block 312, the analysis and post treatment device 102 may determine a post-cosmetic treatment based on the scores for each of the cell characteristics, the cell characteristics, data related to the subject, data related to the cosmetic treatment applied to the biological sample, and/or some other relevant data (such as, for example, the gene expressions classified and grouped into net upregulated and/or downregulated signaling pathways). The analysis and post treatment device 102 may display the post-cosmetic treatment via the user interface 114. In another embodiment, in addition to determining a post-cosmetic treatment, the analysis and post treatment device 102 may list or present the scored and/or determined characteristics of the sample. For example, the analysis and post treatment device 102 may present, via the user interface 114, biological effects (e.g., inflammation, stress, aging, barrier integrity, protein turnover, pigment production, and/or cellular repair, among other biological effects).

Methods 300 and 301 may be an iterative process. For example, a subject or subjects may provide numerous samples and/or different cosmetic treatments may be applied to each one of a different subject and analyzed to determine the most effective and complementary post-cosmetic treatment. For example, a user may provide multiple samples at varying points in time of an ongoing cosmetic treatment. In such an example, each biological sample may be analyzed and a corresponding post-cosmetic treatment may be determined based on the scores for each cell characteristic for each multi-OMICS data, among other factors. Thus, the analysis and post treatment device 102 may determine a post-cosmetic treatment based on data corresponding to a plurality of biological samples.

As described above, example embodiments provide methods and apparatuses that enable improved, complementary, quickly obtained, and effective post-cosmetic treatments. In some embodiments, the post-cosmetic treatment may include a personalized cosmetic treatment regimen. Example embodiments thus provide tools that overcome the problems faced by a user determining a post-cosmetic treatment with limited data, which requires significant expertise, while potentially including less than effective treatments. As these examples all illustrate, example embodiments contemplated herein provide technical solutions that solve real-world problems faced during interpretation of cell analysis.

FIG. 3A and FIG. 3B illustrates operations performed by apparatuses, methods, and computer program products according to various example embodiments. It will be understood that each flowchart block, and each combination of flowchart blocks, may be implemented by various means, embodied as hardware, firmware, circuitry, and/or other devices associated with execution of software including one or more software instructions. For example, one or more of the operations described above may be embodied by software instructions. In this regard, the software instructions which embody the procedures described above may be stored by a memory of an apparatus employing an embodiment of the present invention and executed by a processor of that apparatus. As will be appreciated, any such software instructions may be loaded onto a computing device or other programmable apparatus (e.g., hardware) to produce a machine, such that the resulting computing device or other programmable apparatus implements the functions specified in the flowchart blocks. These software instructions may also be stored in a computer-readable memory that may direct a computing device or other programmable apparatus to function in a particular manner, such that the software instructions stored in the computer-readable memory produce an article of manufacture, the execution of which implements the functions specified in the flowchart blocks. The software instructions may also be loaded onto a computing device or other programmable apparatus to cause a series of operations to be performed on the computing device or other programmable apparatus to produce a computer-implemented process such that the software instructions executed on the computing device or other programmable apparatus provide operations for implementing the functions specified in the flowchart blocks.

The flowchart blocks support combinations of means for performing the specified functions and combinations of operations for performing the specified functions. It will be understood that individual flowchart blocks, and/or combinations of flowchart blocks, can be implemented by special purpose hardware-based computing devices which perform the specified functions, or combinations of special purpose hardware and software instructions.

In some embodiments, some of the operations above may be modified or further amplified. Furthermore, in some embodiments, additional optional operations may be included. Modifications, amplifications, or additions to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A method for determining post-cosmetic treatment, the method comprising:
receiving a biological sample from a subject corresponding to an area treated with a cosmetic treatment;
performing multi-OMICS testing on the biological sample to generate multi-OMICS data;
analyzing the multi-OMICS data, thereby to generate cell characteristics of the biological sample;
determining a difference between the cell characteristics of the biological sample and cell characteristics of a control sample;
scoring at least one cell characteristic of the biological sample based on (a) the difference and (b) on variables within the at least one cell characteristic of the biological sample; and
determining the post-cosmetic treatment based on a score of the at least one cell characteristic.

2. The method of claim 1, wherein the cosmetic treatment comprises use of one or more of a professional device, a home-use device, or a topical treatment.

3. The method of claim 1, wherein the multi-OMICS testing comprises one or more of histology and immunohistochemistry, spatial transcriptomics, bulk RNA sequencing, or protein expression analysis.

4. The method of claim 3, wherein analyzing the multi-OMICS data comprises one or more of overlaying spatial gene expressions with histology images; performing differential expression analysis between the biological sample and the control; analyzing gene ontology and pathway enrichment; or integrating transcriptomics, proteomics, and clinical data.

5. The method of claim 1, wherein analysis of the multi-OMICS data comprises:
applying the multi-OMICS data to a trained machine learning model thereby to predict treatment outcomes.

6. An apparatus for determining post-cosmetic treatment, the apparatus comprising:
A multi-OMICS testing circuitry configured to:
receive a biological sample from a subject corresponding to an area applied with a cosmetic treatment over a selected time,
execute multi-OMICS testing on the biological sample to generate multi-OMICS data; and
a scoring circuitry configured to:
apply the multi-OMICS data to one or more of (a) one or more analysis algorithms or (b) a trained machine learning model, thereby to generate cell characteristics of the biological sample,
determine a difference between the cell characteristics of the biological sample and cell characteristics of a control,
score at least one cell characteristic of the biological sample based on (a) the cell characteristics of the biological sample and (b) the difference, and
determine the post-cosmetic treatment based on a score of the at least one cell characteristic.

7. The apparatus of claim 6, wherein the score indicates an efficacy of the cosmetic treatment in comparison to the control.

8. The apparatus of claim 6, wherein the scoring circuitry is configured to, prior to generation of a score for each of the cell characteristics of the biological sample, classify cell characteristics into net upregulated or net downregulated signaling pathways; and wherein the score for each of the cell characteristics of the biological sample is further based on classification of the cell characteristics.

9. The apparatus of claim 6, wherein the control comprises one or more of an untreated sample, a standard, or a comparable standard.

10. The apparatus of claim 6, wherein the post-cosmetic treatment comprises a personalized cosmetic treatment regimen.

11. The apparatus of claim 6, wherein each of the cell characteristics correlate to selected cellular attributes, and wherein the cellular attributes comprise one or more of lipid metabolism, extracellular matrix organization, cellular stress response, cellular detoxification, immune responses, or inflammatory response.

12. A computer program product for determining post-cosmetic treatment, the computer program product comprising a non-transitory machine-readable storage medium storing software instructions that, when executed, cause an apparatus to:
execute multi-OMICS testing on a biological sample from a subject corresponding to a cosmetic treatment occurring over a selected time, thereby to generate multi-OMICS data; and
apply the multi-OMICS data to one or more analysis instructions, thereby to generate cell characteristics of the biological sample,
determine a difference between the cell characteristics of the biological sample and cell characteristics of an untreated sample or standard,
score the cell characteristics of the biological sample based on (a) the cell characteristics of the biological sample and (b) the difference, and
determine the post-cosmetic treatment based on a score of at least one of the cell characteristics.

13. The computer program product of claim 12, wherein the one or more analysis instructions comprise one or more of (a) multi-OMICS analysis algorithms or (b) a trained machine learning algorithm.

14. The computer program product of claim 12, wherein post-cosmetic treatment comprises one or more of a use of a selected device or a use of a selected cosmetic.
